(19) United States
(12) Patent Application Publication
(10) Pub. No.: US 2023/0270637 A1
(43) Pub. Date: Aug. 31, 2023

(54) Oral Care Compositions

(71) Applicant: Colgate-Palmolive Company, New York, NY (US)

(72) Inventors: Guisheng PAN, Philadelphia, PA (US); Lin FEI, Kendall Park, NJ (US); Suman CHOPRA, Monroe, NJ (US)

(73) Assignee: Colgate-Palmolive Company, New York, NY (US)

(21) Appl. No.: 18/043,679

(22) PCT Filed: Aug. 30, 2021
(86) PCT No.: PCT/US2021/048172

§ 371 Date: Mar. 1, 2023

Related U.S. Application Data
(60) Provisional application No. 63/073,702, filed on Sep. 2, 2020.

(51) Int. Cl. A61K 8/22; A61K 8/36; A61K 8/86; A61K 8/21; A61Q 11/00

(52) U.S. Cl. CPC A61K 8/22; A61K 8/21; A61K 8/86; A61K 8/361; A61Q 11/00; A61K 2800/31

(57) ABSTRACT

Described herein are nonaqueous oral care compositions comprising an orally acceptable vehicle, a whitening agent; and one or more gelling agents wherein the one or more gelling agents comprises a salt of a fatty acid; along with methods of making and using same.

### CROSS-REFERENCE TO RELATED APPLICATION

This application claims the benefit of priority from U.S. Provisional Application No. 63/073,702, filed Sep. 2, 2020, the contents of which are hereby incorporated herein by reference in their entirety.

### BACKGROUND

Conventional oral care products (e.g., toothpastes, whitening gels, whitening trays, etc.) often utilize whitening agents, such as peroxide, to whiten teeth. While toothpastes including whitening agents have proven to be effective, the whitening agents contained therein are often unstable (e.g., reactive) and subject to degradation or reactivity with other components of the toothpastes. For example, the hydrogen peroxide in whitening toothpastes are often highly reactive with conventional thickeners or gelling agents, thereby reducing the whitening efficacy of the toothpastes.

In view of the foregoing, conventional oral care products may often be provided as a two-component whitening system to separate the hydrogen peroxide from potentially reactive components until the time of use when they may be mixed. While conventional two-component whitening systems have been able to prevent reactivity between the hydrogen peroxide and other components of the toothpastes, the implementation of these two-component whitening systems is cost-prohibitive. Further, the two-component whitening systems may often exhibit decreased mixing efficiency, which results in heterogeneous mixtures.

What is needed, then, are improved single phase oral care compositions including whitening agents, such as peroxide, having increased whitening agent stability.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Applicants have discovered that utilization of certain fatty acid salts is effective as a gelling agent for oral care products, particularly for oral care products containing a whitening agent. Thus, in one embodiment, the invention is a nonaqueous oral care composition comprising an orally acceptable vehicle; a whitening agent; and one or more gelling agents, wherein the one or more gelling agents comprises a monovalent salt of a fatty acid.

In at least one embodiment, the fatty acid is a C₁₂-C₃₂ fatty acid. In a further embodiment, the fatty acid is a saturated fatty acid. In other embodiments, the fatty acid is an unsaturated fatty acid. In certain embodiments, the unsaturated fatty acid has an iodine value of less than 5 or less than 1. In certain embodiments, the unsaturated fatty acid is a C₁₆₋₁₈ fatty acid and has an iodine value of less than 5 or less than 1. In certain embodiments, the fatty acid is selected from lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, linoleic acid, arachidonic acid, palmitoleic acid, oleic acid, and a combination of two or more thereof. In certain embodiments, the fatty acid is selected from palmitic acid, stearic acid, and a combination thereof.

In certain embodiments, the salt of a fatty acid is selected from sodium salt, potassium salt, lithium salt, and a combination of two or more thereof. In certain embodiments, the salt of a fatty acid is selected from sodium palmitate, sodium stearate, potassium palmitate, potassium stearate, and a combination of two or more thereof. In certain embodiments, the one or more gelling agents are present in an amount of from about 1.7 weight % to about 10 weight %, based on the total weight of the oral care composition.

In certain embodiments, the orally acceptable vehicle is selected from glycerin, propylene glycol, polyethylene glycol, and combinations thereof. In certain embodiments, the orally acceptable vehicle further comprises a co-polymer of ethylene oxide and propylene oxide. In certain embodiments, the co-polymer of ethylene oxide and propylene oxide has molecular weight of greater than 5000 Da. In certain embodiments, the co-polymer of ethylene oxide and propylene oxide is present in an amount from 5 wt. % to 20 wt. %, from 5 wt. % to 15 wt. %, or from 5 wt. % to 10 wt. %, based on the total weight of the oral care composition. The orally acceptable vehicle may be present in an amount of from about 5 weight % to about 80 weight %, about 10 weight % to about 75 weight %, about 20 weight % to about 75 weight %, about 30 weight % to about 75 weight %, about 40 weight % to about 75 weight %, about 50 weight % to about 75 weight %, about 50 weight % to about 70 weight %, or about 50 weight % to about 65 weight %, based on the total weight of the oral care composition.

In certain embodiments, the whitening agent comprises peroxide. The peroxide whitening agent may comprise hydrogen peroxide or one or more sources of hydrogen peroxide. In certain embodiments, the peroxide whitening agent comprises at least one of hydrogen peroxide, a cross-linked PVP hydrogen peroxide complex, peroxides of alkali and alkaline earth metals, organic peroxy compounds, peroxy acids, pharmaceutically acceptable salts thereof, or mixtures thereof. In certain embodiments, the peroxide whitening agent is a cross-linked PVP hydrogen peroxide complex.

In other embodiments, the whitening agent comprises a non-peroxy compound. In certain embodiments, the non-peroxy compound is selected from the group consisting of metal chlorites, perborates, percarbonates, peroxyacids, hypochlorites, and combinations thereof.

The whitening agent may be present in an amount of from about 1 weight % to about 10 weight %, about 1 weight % to about 7 weight %, about 3 weight % to about 7 weight %, or about 4 weight % to about 6 weight %, based on the total weight of the oral care composition.

In further embodiments, the oral care composition further comprises a fluoride ion source. In certain embodiments, the fluoride ion source is selected from stannous fluoride, sodium fluoride, potassium fluoride, sodium monofluorophosphate, fluorosilicate salts, such as sodium fluorosilicate and ammonium fluorosilicate, amine fluoride, ammonium fluoride, and combinations thereof. In certain embodiments, the fluoride ion source is present in an amount of from about 0.01 weight % to about 5.0 weight %, 0.01 weight % to about 3.0 weight %, or 0.01 weight % to about 1.0 weight %, based on the total weight of the oral care composition.

In certain embodiments, the oral care composition is substantially free of fatty alcohols. In further embodiments, the oral care composition comprises fatty alcohols in an amount of less than 5.0 weight %, less than 3.0 weight %, less than 1.0 weight %, less than 0.1 weight %, less than 0.05 weight %, less than 0.01 weight %, less than 0.005 weight %, or less than 0.0001 weight %, based on the total weight of the oral care composition.

In certain embodiments, the oral care composition comprises water in an amount of less than 5.0 weight %, less than 3.0 weight %, less than 1.0 weight %, or less than 0.5%, based on the total weight of the oral care composition.

In certain embodiments, the oral care composition further comprises an abrasive selected from sodium metaphosphate, potassium metaphosphate, calcium pyrophosphate, magnesium orthophosphate, trimagnesium orthophosphate, tricalcium phosphate, dicalcium phosphate dihydrate, anhydrous dicalcium phosphate and the like, calcium carbonate, magnesium carbonate, hydrated alumina, silica, zirconium silicate, aluminum silicate including calcined aluminum silicate, polymethyl methacrylate, and combinations thereof. In certain embodiments, the abrasive is present in an amount from about 10 weight % to about 20 weight %, about 12 weight % to about 17 weight % or about 15 weight %, based on a total weight of the oral care composition.

In certain embodiments, the oral care composition further comprises an antioxidant. In certain embodiments, the antioxidant is selected from hydroxyanisole (BHA), butylated hydroxytoluene (BHT), vitamin A, carotenoids, vitamin E, flavonoids, polyphenols, ascorbic acid, herbal antioxidants, chlorophyll, melatonin, and combinations thereof. In certain embodiments, the antioxidant is present in an amount from about 0.01% to about 1%, based on a total weight of the oral care composition.

In certain embodiments, the oral care composition further comprises one or more anionic surfactants. In certain embodiments, the one or more anionic surfactants is selected from sodium lauryl benzene sulfonate, dodecyl sodium sulfoacetate, N-2-ethyl laurate potassium sulfoacetamide), sodium lauryl sarcosinate, sodium lauryl sulfate, and sodium ether lauryl sulfate, and combinations thereof. In certain embodiments, the one or more anionic surfactants are present in an amount from about 0.03% to about 5% by weight, about 0.5% to about 3% by weight, about 1.0% to about 3% by weight, or about 2% to about 3% by weight, based on a total weight of the oral care composition.

In further embodiments, the invention is a method for whitening teeth, comprising contacting a tooth surface of a subject in need thereof, with an oral care composition according to any one of the proceeding embodiments. In certain embodiments, the contacting is performed for a sufficient time to achieve a whitening effect on the tooth surface.

Further areas of applicability of the present invention will become apparent from the detailed description provided hereinafter. It should be understood that the detailed description and specific examples, while indicating the typical embodiments of the invention, are intended for purposes of illustration only and are not intended to limit the scope of the invention.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other applications and methods. It is to be understood that the invention is not limited in its application to the details of any particular embodiment shown. The terminology used herein is for the purpose of description and not to limit the invention, its application, or uses.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, “containing”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range.

Unless otherwise specified, all percentages and amounts expressed herein and elsewhere in the specification should be understood to refer to percentages by weight of the total composition.

According to the present application, use of the term “about” in conjunction with a numeral value refers to a value that may be +/- 5% of that numeral. As used herein, the term “substantially free” is intended to mean an amount less than about 5.0 weight %, less than 3.0 weight %, 1.0 wt.%; preferably less than about 0.5 wt.%, and more preferably less than about 0.25 wt.% of the composition.

Unless defined otherwise, all technical and scientific terms used herein have the same meanings as commonly understood by one of ordinary skill in the art to which this invention belongs. All patents, patent applications, publications, and other references cited or referred to herein are incorporated by reference in their entireties for all purposes. In the event of a conflict in a definition in the present disclosure and that of a cited reference, the present disclosure controls.

The present inventors have surprisingly and unexpectedly discovered that certain fatty acid salts may be used as thickening or gelling agents and are compatible with whitening agents in oral care compositions. Particularly, the present inventors have unexpectedly discovered that conventional thickening agents may be replaced with certain fatty acid salts to provide similar or comparable compatibility. The present inventors have also surprisingly and unexpectedly discovered a method for increasing whitening agent (e.g. peroxide) stability in a single phase oral care composition. The method for increasing whitening agent (e.g. peroxide) stability may include replacing conventional thickening agents with certain fatty acid salts. The method may also include preparing an oral care composition that is free, or substantially free, of fatty alcohols. It should be appreciated that the increased peroxide stability in the oral care compositions may be achieved without encapsulations and/or film-type materials to enhance the stability thereof.

Compositions disclosed herein may be or include an oral care composition. The oral care composition may be a non-aqueous oral care composition, such as a non-aqueous dentifrice or toothpaste. The oral care composition may include an orally acceptable vehicle, a whitening agent, and one or more gelling agents. The gelling agents may be or include, but are not limited to, one or more fatty acid salts. In at least one implementation, the gelling agents are free, or substantially free, of fatty alcohols. As used herein, “substantially free of fatty alcohols” may refer to a composition that contains fatty alcohols in an amount of less than 5.0 weight %, less than 3.0 weight %, less than 1.0 weight %, less than 0.1 weight %, less than 0.05 weight %, less than 0.01 weight %, less than 0.005 weight %, or less than 0.0001 weight %, based on a total weight of the oral care composition. As used herein, “free of fatty alcohols” may refer to a composition that contains less than 0.5 weight %, less than 0.1 weight %, less than 0.05 weight %, less than 0.01 weight %, less than 0.005 weight %, or less than 0.0001 weight %, based on a total weight of the oral care composition.

In certain embodiments, the oral care composition prior to use may be anhydrous. For example, the oral care composition may be free, or substantially free, of water. As used herein, “substantially free of water” may refer to a composition that contains water in an amount of less than 5.0 weight %, less than 3.0 weight %, less than 1.0 weight %, less than 0.1 weight %, less than 0.05 weight %, less than 0.01 weight %, less than 0.005 weight %, or less than 0.0001 weight %, based on a total weight of the oral care composition. As used herein, “free of water” may refer to a composition that contains water in an amount of less than 0.5 weight %, less than 0.1 weight %, less than 0.05 weight %, less than 0.01 weight %, less than 0.005 weight %, or less than 0.0001 weight %, based on a total weight of the oral care composition. In certain embodiments, the oral care composition prior to use may have a “low water content”. As used herein, “low water content” may refer to a composition that contains water in an amount less than about 5 weight %, less than about 7 weight %, or less than about 10 weight %.

It should be appreciated that all ingredients for use in the compositions described herein are orally acceptable. As used herein, the expression “orally acceptable” may define an ingredient that is present in a composition as described in an amount and form that does not render the composition unsafe for use in the oral cavity.

The oral care composition may be a single phase oral care product or single phase oral care composition. For example, all the components of the oral care composition may be maintained together with one another in a single phase and/or vessel. For example, all the components of the oral care composition may be maintained in a single phase, such as a single homogenous phase. The single homogenous phase may be an anhydrous product or an anhydrous composition.

The oral care composition may be or form at least a portion of one or more oral care products. The oral care composition may include or be combined with an orally acceptable vehicle to form the oral care product (e.g., the toothpaste). Illustrative oral care products may include, but are not limited to, a toothpaste (dentifrice), a prophylactic paste, a tooth polish, a tooth gel (e.g., a whitening gel), a chewing gum, a lozenge, a mouthwash, a whitening strip, a paint-on gel, varnish, veneer, and tube, syringe or dental tray comprising a gel or paste, or a gel or paste coated on an application support such as dental floss or a toothbrush (e.g., a manual, electric, sound, a combination thereof or ultrasound toothbrush). In one implementation, the oral care composition may be or may form at least a portion of a toothpaste.

In at least one implementation, the orally acceptable vehicle may be, or include, one or more humectants. Illustrative humectants may be or include, but are not limited to, glycerin, propylene glycol, polyethylene glycol, block copolymers of ethylene oxide (EO) and propylene oxide (PO), and combinations thereof. In a preferred implementation, the orally acceptable vehicle may be or include, but is not limited to, propylene glycol and block copolymers of ethylene oxide (EO) and propylene oxide (PO).

In a preferred implementation, the orally acceptable vehicle may be or include, but is not limited to, propylene glycol. The propylene glycol may be present in an amount of from 5 weight % to about 80 weight %, based on a total weight of the oral care composition. For example, the propylene glycol may be present in an amount of from about 5 weight %, about 10 weight %, about 15 weight %, or about 20 weight % to about 25 weight %, about 30 weight %, about 35 weight %, about 40 weight %, about 45 weight %, about 50 weight %, about 55 weight %, about 60 weight %, about 65 weight %, about 70 weight %, about 75 weight %, or about 80 weight %. In another example, the propylene glycol may be present in an amount of from about 5 weight % to about 80 weight %, about 10 weight % to about 75 weight %, about 20 weight % to about 75 weight %, about 30 weight % to about 75 weight %, about 40 weight % to about 75 weight %, about 50 weight % to about 75 weight %, about 50 weight % to about 70 weight %, or about 50 weight % to about 65 weight %, based on the total weight of the oral care composition. In an exemplary implementation, the propylene glycol may be present in an amount of about 50 weight % to about 75 weight %, preferably about 55 weight % to about 70 weight %, and more preferably about 55 weight % to about 65 weight %, based on the total weight of the oral care composition. In a preferred implementation, the propylene glycol may be present in an amount of about 55 weight % to about 65 weight % or about 60 weight %.

In some embodiments, the composition further comprises an ethylene oxide, propylene oxide block co-polymer. In certain embodiments, the block copolymers of ethylene oxide and propylene oxide may be represented by formula (1); (ethylene oxide)ₓ-(propylene oxide)_{y}-(ethylene oxide)_{z}; where x may be an integer of from about 5 to about 15 (e.g., x=9-13, or about 11), y may be an integer from about 10 to about 20 (e.g., y=13-17, or about 16), and z may be an integer from about 5 to about 15 (e.g., x=9-13, or about 11). In another embodiment, x may be an integer from about 2 to about 65, y may be an integer from about 15 to about 70, and z may be an integer from about 2 to about 65. In certain embodiments, the block copolymer of ethylene oxide and propylene oxide may be represented by formula (2); (ethylene oxide)₁₁-(propylene oxide)₁₆-(ethylene oxide)₁₁. The block copolymer of ethylene oxide and propylene oxide may have an average molecular weight greater than or equal to about 1,000 Da and less than or equal to about 3,000 Da. For example, the block copolymer of ethylene oxide and propylene oxide may have an average molecular weight of from about 1,000 Da, about 1,100 Da, about 1,200 Da, about 1,300 Da, about 1,400 Da, about 1,500 Da, about 1,600 Da, about 1,700 Da, about 1,800 Da, or about 1,850 Da to about 1,950 Da, about 2,000 Da, about 2,100 Da, about 2,200 Da, about 2,300 Da, about 2,400 Da, about 2,500 Da, about 2,600 Da, about 2,700 Da, about 2,800 Da, about 2,900 Da, or about 3,000 Da. In another example, the block copolymer of ethylene oxide and propylene oxide may have an average molecular weight of from about 1,000 Da to about 2,800 Da, about 1,100 Da to about 2,700 Da, about 1,200 Da to about 2,600 Da, about 1,300 Da to about 2,500 Da, about 1,400 Da to about 2,400 Da, about 1,500 Da to about 2,300 Da, about 1,600 Da to about 2,200 Da, about 1,700 Da to about 2,100 Da, about 1,800 Da to about 2,000 Da, or about 1,850 Da to about 1,950 Da. In an exemplary implementation, the block copolymer of ethylene oxide and propylene oxide may have an average molecular weight of about 1,850 Da to about 1,950 Da, preferably about 1,900 Da.

In certain embodiments, the ethylene oxide, propylene oxide block co-polymer may be represented by Formula (3); (ethylene oxide)ₓ-(propylene oxide)_{y} wherein x is an integer of 80-150 and y is an integer 30-80. Optionally, the ethylene oxide, propylene oxide block co-polymer is present in an amount of from 5 wt. % to 12 wt. %, based on the weight of the composition. In some embodiments, the composition comprises an ethylene oxide, propylene oxide block co-polymer of formula (ethylene oxide)ₓ-(propylene oxide)_{y} wherein x is an integer of 80-150, e.g. 100-130, e.g. about 118, and y is an integer 30-80, e.g. about 60-70, e.g. about 66, having an average molecular weight of copolymer of from about 2,000 Da to about 15,000 Da. In certain embodiments, the molecular weight of copolymer has an average molecular weight of greater than 5000 Da, e.g., 8000-13000 Da, e.g. about 9800 Da. In some embodiments, the composition comprises an ethylene oxide, propylene oxide block co-polymer of average molecular weight greater than 5000 Da, being substantially free of an ethylene oxide, propylene oxide block co-polymer of average molecular weight less than 5000 Da. Block copolymers of ethylene oxide/propylene oxide are useful, but higher molecular weight, e.g., >5000 Da are preferred, e.g. including Pluracare^{®} L1220 (available from BASF, Wyandotte, Mich., United States of America). Further illustrative block copolymers of ethylene oxide (EO) and propylene oxide (PO) may be or include, but are not limited to, PLURONIC® L1, PLURONIC® L43, PLURONIC® L10, PLURONIC® L44, PLURONIC® 10R5, PLURONIC® 17R4, PLURONIC® L25R4, PLURONIC® P84, PLURONIC® P65, PLURONIC® P104, PLURONIC® P105, and the like, and combinations thereof, all of which are commercially available from BASF of Mount Olive, N.J.

In certain embodiments, the ethylene oxide, propylene oxide block co-polymer is present in an amount from 5 wt. % to 20 wt. %, from 5 wt. % to 15 wt. %, or from 5 wt. % to 10 wt. %, based on the total weight of the oral care composition. In certain embodiments, the ethylene oxide, propylene oxide block co-polymer is present up to 7 wt. %, up to 10 wt. %, up to 13 wt. %, up to 16 wt. %, or up to 20 wt. %, based on the total weight of the oral care composition.

The orally acceptable vehicle or the humectant thereof (e.g., propylene glycol and/or ethylene oxide, propylene oxide block co-polymer) may be present in an amount of from 5 weight % to about 80 weight %, based on a total weight of the oral care composition. For example, the orally acceptable vehicle or the humectant thereof may be present in an amount of from about 5 weight %, to about 80 weight %, from about from about 20 weight % to about 80 weight %, from about 40 weight % to about 80 weight %, from about 50 weight % to about 80 weight %, from about 55 weight % to about 75 weight %, or from about 55 weight % to about 70 weight %, based on the total weight of the oral care composition. In another example, the orally acceptable vehicle or the humectant thereof may be present in an amount of from about 55 weight % to about 80 weight %, from about 60 weight % to about 75 weight %, or from about 65 weight % to about 75 weight %. In another example, the orally acceptable vehicle or the humectant thereof may be present in an amount of from about 30 weight % to about 80 weight %, from about 40 weight % to about 80 weight %, from about 50 weight % to about 80 weight %, or from about 60 weight % to about 80 weight %. In an exemplary implementation, the orally acceptable vehicle or the humectant thereof may be present in an amount of from about 50 weight % to about 80 weight %, preferably from about 55 weight % to about 70 weight %, and more preferably from about 60 weight % to about 70 weight %. In a preferred implementation, the orally acceptable vehicle or the humectant thereof may be present in an amount of from about 55 weight % to about 80 weight %, or about 72 weight %.

The oral care composition may include one or more whitening agents. In certain embodiments, the one or more whitening agents comprise peroxide. In certain embodiments, the one or more whitening agents comprise a non-peroxy compound.

As described above, the oral care composition includes one or more whitening agents. As used herein, a “whitening agent” is a material which effects whitening of a tooth surface to which it is applied. For example, in some embodiments, the whitening agent is an oxidizing agent. In its broadest sense, “oxidizing agent” is intended to include those compounds which can accept an electron from another molecule in the environment of the oral cavity without having a deleterious or unacceptably harmful effect on the oral cavity in normal and accepted use.

In some embodiments, the whitening agent may include peroxide. In certain embodiments, the whitening agent may include hydroperoxides, such as hydrogen peroxide, peroxides of alkali and alkaline earth metals, organic peroxy compounds, peroxy acids, salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include urea peroxide, carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts include organic peroxy acids such as alkyl peroxy acids, and monoperoxyphthalate and mixtures thereof, as well as inorganic peroxy acid salts such as percarbonate, perphosphate, perborate and persilicate salts of alkali and alkaline earth metals such as lithium, potassium, sodium, magnesium, calcium and barium, and mixtures thereof.

The peroxide whitening agents may be or include, but are not limited to, hydrogen peroxide or one or more sources of hydrogen peroxide. For example, the peroxide whitening agents may be hydrogen peroxide and/or hydrogen peroxide releasing substances. The one or more sources of hydrogen peroxide may be or include any compound or material configured to release hydrogen peroxide. Preferably, the peroxide whitening agents include, but are not limited to, solid peroxide whitening agents and bound peroxide whitening agents that are substantially anhydrous oxygen generating compounds. Solid peroxide whitening agents may include, but are not limited to, peroxides and persulfates. Exemplary peroxide agents include hydroperoxides, hydrogen peroxide, peroxides of alkali and alkaline earth metals, organic peroxy compounds, peroxy acids, pharmaceutically-acceptable salts thereof, and mixtures thereof. Peroxides of alkali and alkaline earth metals include, but are not limited to, lithium peroxide, potassium peroxide, sodium peroxide, magnesium peroxide, calcium peroxide, barium peroxide, and mixtures thereof. Organic peroxy compounds include, but are not limited to, urea peroxide, glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts include, but are not limited to, organic peroxy acids such as alkyl peroxy acids, and monoperoxyphthalate and mixtures thereof, as well as inorganic peroxy acid salts such as percarbonate and perborate salts of alkali and alkaline earth metals such as lithium, potassium, sodium, magnesium, calcium and barium, and mixtures thereof. Preferred solid peroxides are sodium perborate, urea peroxide, and mixtures thereof.

The peroxide whitening agents may be preferably bound. For example, peroxide may be bound to a polymer such as PVP (poly(N-vinylpyrrolidone). Suitable PVP complexes are disclosed, for example, in U.S. Pat. No. 5,122,370, the contents of which are incorporated herein by reference. In some implementations, it may be desirable to use any known peroxide whitening agent except sodium percarbonate and/or any of the percarbonate salts. The sources of hydrogen peroxide or peroxide whitening agents may also be or include, but are not limited to, PEROXYDONE™ XL 10 complex or POLYPLASDONE® XL 10F, which are commercially available from Ashland Inc. of Covington, Ky. In a typical implementation, the source of hydrogen peroxide includes a cross-linked PVP hydrogen peroxide complex.

In some embodiments a non-peroxide whitening agent may be used. Whitening agents among those useful herein include non-peroxy compounds, such as chlorine dioxide, chlorites and hypochlorites. Chlorites and hypochlorites include those of alkali and alkaline earth metals such as lithium, potassium, sodium, magnesium, calcium and barium. Non-peroxide whitening agents also include colorants, such as titanium dioxide and hydroxyapatite. In certain embodiments, the non-peroxy whitening agent is selected from the group consisting of metal chlorites, perborates, percarbonates, peroxyacids, hypochlorites, and combinations thereof.

In certain embodiments, the whitening agent comprises both a peroxide and a non-peroxy whitening agent.

The amount or concentration of the source of whitening agent may vary widely. In some embodiments, the oral care composition includes from about 0.01% to about 50% whitening agent based on a total weight of the oral care composition. In other embodiments, the oral care composition includes from about 0.05 weight % to about 40 weight % whitening agent. In one embodiment, the oral care composition includes about 5.5 weight % whitening agent based on a total weight of the oral care composition. In at least one example, the one or more whitening agents may be present in an amount that provides a concentration of hydrogen peroxide of less than or equal to 4 weight %, less than or equal to 3.5 weight %, less than or equal to 3 weight %, less than or equal to 2.5 weight %, less than or equal to 2 weight %, or less than or equal to 1.5 weight %, based on a total weight of the oral care composition. In at least one implementation, the source of hydrogen peroxide may be present in an amount greater than or equal to 1 weight % and less than or equal to 30 weight %, based on a total weight of the oral care composition. For example, the source of hydrogen peroxide may be present in an amount of from about 1 weight %, about 3 weight %, about 5 weight %, about 7 weight %, about 9 weight %, about 11 weight %, or about 13 weight %. In other embodiments, the source of hydrogen peroxide may be present in an amount of from about 1 weight % to about 10 weight %, about 1 weight % to about 7 weight %, about 3 weight % to about 7 weight %, or about 4 weight % to about 6 weight %, based on the total weight of the oral care composition. In another example, the source of hydrogen peroxide may be present in an amount of from about 1 weight % to about 30 weight %, about 3 weight % to about 29 weight %, about 5 weight % to about 27 weight %, about 7 weight % to about 25 weight %, about 9 weight % to about 23 weight %, about 11 weight % to about 21 weight %, about 13 weight % to about 19 weight %, or about 15 weight % to about 17 weight %. In a preferred implementation, the source of hydrogen peroxide is a cross-linked PVP complexed with hydrogen peroxide, and is present in an amount of from about 2 weight % to about 10 weight %, preferably about 4 weight % to about 8 weight %, and more preferably about 5.5 weight %.

The oral care composition may include one or more thickening or gelling agents capable of or configured to thicken the oral care composition. Illustrative gelling agents may also be or include, but are not limited to, one or more salts of a fatty acid. As used herein, the term “salt of a fatty acid” refers to an aliphatic monocarboxylic acid whose carboxylic acid functional group is in the form of a salt. The hydrocarbon chain of the fatty acid salt may be saturated or unsaturated (e.g., alkyl, alkenyl or alkynyl hydrocarbon chains). In addition, the hydrocarbon chain may be straight or branched. Moreover, in some embodiments, hydrogens in the hydrocarbon chain may be substituted. In preferred embodiments, the fatty acid is a C₁₂-C₃₂ fatty acid.

The fatty acid may be or include an unsaturated linear and/or a saturated linear fatty acid. In at least one implementation, the fatty acid may be or include one or more unsaturated linear or saturated linear C₁₂-C₃₂ fatty acids. It should be appreciated that the oral care composition may include any one or more fatty acids within the indicated carbon number range. For example, the gelling agents in the oral care composition may be or include salts of lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, linoleic acid, arachidonic acid, palmitoleic acid, oleic acid, and the like, and mixtures or combinations thereof. In an exemplary implementation the oral care composition includes salts of palmitic acid and/or stearic acid as thickening agents.

Unsaturated fatty acids useful in the invention may be almost, but not fully hydrogenated. The amount of hydrogenation may be measured by determining the iodine value. The iodine value can be measured by ASTM D5554-95 (2006). In certain embodiments, the unsaturated fatty acid is a C₁₂-C₃₂ fatty acid. In certain embodiments, unsaturated fatty acid has an iodine value of less than 20. In certain embodiments, unsaturated fatty acid has an iodine value of less than 10. In certain embodiments, unsaturated fatty acid has an iodine value of less than 5. In certain embodiments, unsaturated fatty acid has an iodine value of less than 1. In further embodiments, the unsaturated fatty acid is a C₁₆₋₁₈ fatty acid and has an iodine value of less than 20, 10, 5, or less than 1.

Embodiments of the invention utilize the salt of fatty acid, wherein the salt comprises a monovalent salt. In preferred embodiments, the salt of fatty acid comprises a sodium salt. In other embodiments, the salt of fatty acid comprises a potassium salt. In other embodiments, the salt of fatty acid comprises a lithium salt. In other embodiments, the salt of fatty acid comprises salt selected from sodium salt, potassium salt, lithium salt, and a combination of two or more thereof. In other embodiments, the salt of fatty acid is a combination of sodium and potassium salt. In certain embodiments, the salt of fatty acid comprises sodium palmitate. In certain embodiments, the salt of fatty acid comprises sodium stearate. In certain embodiments, the salt of fatty acid comprises potassium palmitate. In certain embodiments, the salt of fatty acid comprises potassium stearate. In certain embodiments, the salt of fatty acid is selected from sodium palmitate, sodium stearate, potassium palmitate, potassium stearate, and a combination of two or more thereof.

The amount or concentration of the thickening agents may vary widely. In at least one example, the thickening agents may be present in an amount greater than or equal to 0.1 weight % and less than or equal to 50 weight %, based on the total weight of the oral care composition. For example, the thickening agents may be present in an amount of from about 0.1 weight %, about 0.2 weight %, about 0.4 weight %, about 0.6 weight %, about 0.8 weight %, about 1 weight %, about 1.5 weight %, about 2 weight %, about 2.5 weight %, or about 3 weight % to about 3.5 weight %, about 4 weight %, about 4.5 weight %, about 5 weight %, about 5.5 weight %, about 6.0 weight %, about 6.5 weight %, about 7.0 weight %, about 7.5 weight %, about 8.0 weight %, about 8.5 weight %, about 9.0 weight %, about 9.5 weight %, or about 10.0 weight %, based on the total weight of the oral care composition. In another example, the thickening agents may be present in an amount of from about 1 weight % to about 10 weight %, about 1.5 weight % to about 9.5 weight %, about 2 weight % to about 8 weight %, about 2.5 weight % to about 7.5 weight %, or about 3 weight % to about 7 weight %. In another example, the thickening agents may be present in an amount of from about 1 weight %, about 2 weight %, about 3 weight %, about 4 weight %, about 5 weight %, about 6 weight %, about 7 weight %, about 8 weight %, about 9 weight %, or about 10 weight % to about 10 weight %, about 20 weight %, about 25 weight %, about 30 weight %, about 35 weight %, about 40 weight %, about 45 weight %, or about 50 weight %, based on the total weight of the oral care composition. In a preferred embodiment, the one or more thickening agents may be present in an amount of from about 1 weight % to about 10 weight %, about 2 weight % to about 8 weight %, about 3 weight % to about 7 weight %, based on the total weight of the oral care composition.

In at least one implementation, the oral care composition may be free or substantially free of fluoride (e.g., soluble fluoride salts). In another implementation, the oral care composition may further include fluoride, such as one or more fluoride ion sources (e.g., soluble fluoride salts). A wide variety of fluoride ion-yielding materials may be employed as sources of soluble fluoride. Examples of suitable fluoride ion-yielding materials may be found in U.S. Pat. No. 3,535,421 to Briner et al., U.S. Pat. No. 4,885,155 to Parran, Jr. et al., and U.S. Pat. No. 3,678,154 to Widder et al., the disclosures of which are incorporated herein by reference. Illustrative fluoride ion sources include, but are not limited to, fluoride, stannous fluoride, sodium fluoride, potassium fluoride, sodium monofluorophosphate, fluorosilicate salts, such as sodium fluorosilicate and ammonium fluorosilicate, amine fluoride, ammonium fluoride, and combinations thereof. In a typical implementation, the fluoride ion source includes sodium monofluorophosphate. The amount of the fluoride ion source in the oral care composition may be greater than 0 weight % and less than 0.9 wt %, less than 0.8 wt %, less than 0.7 wt %, less than 0.6 wt %, or less than 0.5 wt %. In some embodiments, the fluoride ion source is present in an amount of from about 0.01 weight % to about 5.0 weight %, 0.01 weight % to about 3.0 weight %, or 0.01 weight % to about 1.0 weight %, based on the total weight of the oral care composition. The fluoride ion sources may be present in an amount sufficient to provide a total of about 100 to about 20,000 ppm, about 200 to about 5,000 ppm, or about 500 to about 2,500 ppm fluoride ions.

The oral care composition may include an abrasive system including one or more abrasives. As used herein, the term “abrasive” may also refer to materials commonly referred to as “polishing agents”. Illustrative abrasives may include, but are not limited to, phosphate salts (e.g., insoluble phosphate salts), such as sodium metaphosphate, potassium metaphosphate, calcium pyrophosphate, magnesium orthophosphate, trimagnesium orthophosphate, tricalcium phosphate, dicalcium phosphate dihydrate, anhydrous dicalcium phosphate and the like, calcium carbonate, magnesium carbonate, hydrated alumina, silica, zirconium silicate, aluminum silicate including calcined aluminum silicate, polymethyl methacrylate, and the like, and mixtures or combinations thereof.

Illustrative abrasives may also be or include, but are not limited to, those previously considered to be incompatible in a peroxide containing formulation (“a peroxide-incompatible abrasive”). As used herein, “a peroxide-incompatible abrasive” may refer to an abrasive that substantially reacts with hydrogen peroxide in an aqueous medium (e.g., solution) so as to reduce a whitening efficacy of the medium. “A peroxide-incompatible abrasive” may also refer to an abrasive that reacts with hydrogen peroxide in a single phase oral care composition (e.g., toothpaste) such that the amount of hydrogen peroxide present in the oral care composition after exposure to accelerated aging conditions for a period of 1, 2, 3, 4, 5, 10, 15, or 20 weeks is reduced by at least 0.5%, at least 0.6%, at least 0.7%, at least 0.8%, at least 0.9%, at least 1.0%, at least 1.1%, at least 1.2%, at least 1.3%, at least 1.4%, at least 1.5%, at least 1.6%, at least 1.7%, at least 1.8%, at least 1.9%, at least 2.0%, at least 2.5%, at least 3.0%, at least 3.5%, at least 4.0%, at least 4.5%, at least 5.0%, at least 5.5%, at least 6.0%, at least 6.5%, at least 7.0%, at least 7.5%, at least 8.0%, at least 8.5%, at least 9.0%, at least 9.5%, at least 10.0%, at least 10.5%, at least 11.0%, at least 11.5%, at least 12.0%, at least 12.5%, at least 13.0%, at least 13.5%, at least 14.0%, at least 14.5%, or at least 15%. Illustrative “peroxide-incompatible abrasives” may be or include, but are not limited to, silica, dicalcium phosphate hydrate, calcium carbonate, hydroxyapatite, calcium phosphate, and the like.

The amount of the abrasive system and abrasives thereof may vary widely. In at least one implementation, the amount of the abrasives may be from greater than 0 weight % to about 40 weight %, based on the total weight of the oral care composition. For example, the amount of the abrasives present in the oral care composition may be from greater than 0 weight %, about 2 weight %, about 4 weight %, about 6 weight %, about 8 weight %, about 10 weight %, about 12 weight %, about 14 weight %, about 16 weight %, about 18 weight %, or about 19 weight % to about 21 weight %, about 22 weight %, about 24 weight %, about 26 weight %, about 28 weight %, about 30 weight %, about 32 weight %, about 34 weight %, about 36 weight %, about 38 weight %, or about 40 weight %. In another example, the amount of the abrasives present in the oral care composition may be from greater than 0 weight % to about 40 weight %, about 2 weight % to about 38 weight %, about 4 weight % to about 36 weight %, about 6 weight % to about 34 weight %, about 8 weight % to about 32 weight %, about 10 weight % to about 30 weight %, about 12 weight % to about 28 weight %, about 14 weight % to about 26 weight %, about 16 weight % to about 24 weight %, about 18 weight % to about 22 weight %, or about 19 weight % to about 21 weight %. In a preferred implementation, the abrasives present in the oral care composition may be from about 10 weight % to about 20 weight %, preferably about 12 weight % to about 17 weight %, or more preferably about 15 weight %, based on a total weight of the oral care composition.

It should be appreciated to one having ordinary skill in the art, that the oral care composition may include other additional ingredients/components. For example, the oral care composition may include anti-caries agents, desensitizing agents, viscosity modifiers, diluents, pH modifying agents, mouth feel agents, sweetening agents, flavor agents, colorants, preservatives, and the like, and combinations and mixtures thereof. It should further be appreciated by one having ordinary skill in the art that while general attributes of each of the above categories of materials may differ, there may be some common attributes and any given material may serve multiple purposes within two or more of such categories of materials.

In at least one implementation, the additional ingredients/components may include one or more active materials configured to prevent and/or treat one or more conditions and/or disorders of the oral cavity. For example, the one or more active materials may be configured to prevent and/or treat one or more conditions and/or disorders of hard and/or soft tissue of the oral cavity. The active materials may also be configured to prevent and/or treat one or more physiological disorders and/or conditions, and/or provide a cosmetic benefit to the oral cavity.

In at least one implementation, the oral care composition may include an anticalculus agent. Generally, anticalculus agents may not be compatible with some oral care compositions; however, implementations of the present disclosure may incorporate anticalculus agents and the oral care ingredients into a single phase oral care composition. Illustrative anticalculus agents may include, but are not limited to, phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates. In a typical implementation, the anticalculus agents include tetrasodium pyrophosphate (TSPP), sodium tripolyphosphate (STPP), or a combination thereof.

In certain embodiments, the oral care composition may include comprise an effective amount of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, disodium hydrogenorthophoshpate, monosodium phosphate, pentapotassium triphosphate and mixtures of any of two or more of these, e.g., in an amount of 0.01-20%, e.g., 0.1-8%, e.g., e.g., 0.1 to 5%, e.g., 0.3 to 2%, e.g., 0.3 to 1%, e.g. about 0.01%, about 0.1%, about 0.5%, about 1%, about 2%, about 5%, about 6%, by weight of the composition. In certain embodiments, the alkali phosphate salt is selected from tetrapotassium pyrophosphate, disodium hydrogenorthophoshpate, monosodium phosphate, and pentapotassium triphosphate, and combinations thereof. In certain embodiments, the alkali phosphate salt is a polyphosphate. In certain embodiments, the polyphosphate is tetrasodium pyrophosphate. In certain embodiments, the tetrasodium pyrophosphate is present in an amount from 0.5-2.0 wt. % (e.g., about 1.5 wt. %), by weight of the composition.

The oral care compositions of the invention may also include a flavoring agent. Flavoring agents which are used in the practice of the present invention include, but are not limited to, essential oils and various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint. The flavoring agent is incorporated in the oral composition at a concentration of 0.01 to 2%, by weight of the composition.

Sweeteners among those useful herein include orally acceptable natural or artificial, nutritive or non-nutritive sweeteners. Such sweeteners include dextrose, polydextrose, sucrose, maltose, dextrin, dried invert sugar, mannose, xylose, ribose, fructose, levulose, galactose, corn syrup (including high fructose corn syrup and corn syrup solids), partially hydrolyzed starch, hydrogenated starch hydrolysate, sorbitol, mannitol, xylitol, maltitol, isomaltose, aspartame, neotame, saccharin and salts thereof, sucralose, dipeptide-based intense sweeteners, cyclamates, dihydrochalcones and mixtures thereof. Some embodiments may include one or more sweeteners. In some embodiments, the oral care composition includes from about 0.005% to about 5% of one or more sweeteners, based on a total weight of the oral care composition. In other embodiments, the oral care composition includes from about 0.01% to about 1% of one or more sweeteners, based on a total weight of the oral care composition.

The oral care composition may include an antioxidant. Any orally acceptable antioxidant may be used, including, but not limited to, butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and the like, and combinations and mixtures thereof. In some embodiments, the oral care composition includes from about 0.005% to about 5% of one or more antioxidants, based on a total weight of the oral care composition. In other embodiments, the oral care composition includes from about 0.01% to about 1% of one or more antioxidants, based on a total weight of the oral care composition.

In certain embodiments, oral care composition contains one or more anionic surfactants, for example, water-soluble salts of higher fatty acid monoglyceride monosulfates, such as the sodium salt of the monosulfated monoglyceride of hydrogenated coconut oil fatty acids, such as sodium N- methyl N-cocoyl taurate or sodium cocomo-glyceride sulfate; higher alkyl sulfates, such as sodium lauryl sulfate; higher alkyl-ether sulfates, e.g., of formula CH₃(CH₂)ₘCH₂(OCH₂CH₂)ₙOSO₃X, wherein m is 6-16, e.g., 10, n is 1-6, e.g., 2, 3 or 4, and X is Na or K, for example sodium laureth-2 sulfate (CH₃(CH₂)₁₀CH₂(OCH₂CH₂)₂OSO₃Na); higher alkyl aryl sulfonates such as sodium dodecyl benzene sulfonate (sodium lauryl benzene sulfonate); higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. As used herein, “higher alkyl” may refer to a C₆-C₃₀ alkyl. In particular embodiments, the anionic surfactant (where present) is selected from sodium lauryl sulfate (SLS) and sodium ether lauryl sulfate. When present, the anionic surfactant is present in an amount which is effective, e.g., >0.001% by weight of the formulation. In one embodiment, the one or more anionic surfactants are present in an amount from about 0.03% to about 5% by weight, about 0.5% to about 3% by weight, about 1.0% to about 3% by weight, or about 2% to about 3% by weight, based on a total weight of the oral care composition.

The present disclosure may provide methods for increasing whitening agent (e.g. peroxide) stability in an oral care composition. The method may include inclusion of one or more gelling agents, wherein the gelling agents comprise a salt of a fatty acid into an oral care composition comprising an orally acceptable vehicle and a peroxide whitening agent. Such methods may yield maintaining viability, stability, and/or compatibility with the peroxide whitening agent for at least three months.

While ingredients are sometimes identified herein by category, e.g., humectant, antioxidant, thickener, etc., this identification is for convenience and clarity, but is not intended to be limiting. All of the ingredients in the compositions may have functions in addition to their primary function, and may contribute to the overall properties of the composition, including its stability, efficacy, consistency, mouthfeel, taste, odor and so forth.

The present disclosure also provides methods to whiten an oral surface in a human or animal subject comprising contacting an oral care whitening composition described herein with the oral surface. The oral surface is preferably teeth. As used herein “animal subject” may include higher order non-human mammals such as canines, felines, and horses. In some embodiments, contacting the surface of the teeth with the oral care whitening composition may include disposing the oral care whitening composition on a surface of an implement, such as a toothbrush, and contacting the whitening composition with the surface of the teeth. The oral care whitening composition may be applied to the teeth and left for at least 1 minute, 2 minutes, or 5 minutes. In some embodiments, contacting the surface of the teeth with the oral care whitening composition may include disposing the oral care whitening composition in a dental tray (e.g., reservoir of the dental tray) and disposing the dental tray about the teeth. The dental tray may be applied to the teeth and left for at least 2 minutes, at least 5 minutes, typically at least 10 minutes, or more typically at least 30 minutes. After each treatment with the tooth oral care whitening composition the teeth may be treated with a tooth desensitizing formulation. Illustrative desensitizing formulations may contain potassium nitrate, citric acid, citric acid salts, strontium chloride and the like.

The oral care whitening composition may be applied and/or contacted with the surfaces of the teeth at predetermined intervals. For example, a daily basis, at least once a day for multiple days, or alternatively every other day. In another example, the oral care whitening composition may be applied and/or contacted with the surfaces of the teeth at least once a day, at least once every two days, at least once every three days, at least once every five days, at least once a week, at least once every two weeks, or at least once a month. The oral care whitening composition may be utilized for up to 2 weeks, up to 3 weeks, up to 4 weeks, up to 6 weeks, up to 8 weeks, or greater.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

An oral care composition was prepared by combining the ingredients/components according to Table 1. Briefly, the sodium fatty acid source contained mixed fatty acids (mostly stearate with a mass stearic acid to palmitic acid ratio of up to about 50%/50%) were first dissolved in propylene glycol (PG) and L1220 at elevated temperature (65-70° C.) and then mixed with an abrasive, such as calcium pyrophosphate, and minor solids (sodium monofluorophosphate (MFP), saccharin, and sucralose) for 20 minutes. Next, PVP-H₂O₂ complex polymer was added and mixed for 20 minutes. Finally, SLS and flavor were added and mixed for 7 minutes. The resulted toothpaste appeared as a smooth white paste. Initial Brookfield viscosity was 993,435 cP and 10% pH in water was 8.94.

TABLE 1

| Material | Quantity (wt. % of total composition) |
|---|---|
| Propylene Glycol | 61.26 |
| Co-polymer of ethylene oxide + propylene oxide | 10 |
| Sodium fatty acid | 3.5 |
| Sodium Monofluorophosphate | 0.76 |
| Sweetener | 0.65 |
| PVP-H₂O₂ | 5.5 |
| Abrasive | 15 |
| Flavor | 1.8 |
| Antioxidant | 0.03 |
| Surfactant | 1.5 |

### Example 2

The example composition in Table 1 was further analyzed for viscosity change over time. The results are summarized in Table 2. As shown in Table 2, the viscosity stabilized within one week.

TABLE 2

| | | | | |
|---|---|---|---|---|
| Day | 0 | 1 | 5 | 8 |
| Viscosity (CP) | 993,435 | 1,280,009 | 1,546,920 | 1,055,410 |

### Example 3

Rapid bloating testing at 60° C. was performed for one week. The stability of the composition of Table 1 was compared to a toothpaste control having similar H₂O₂ and fatty alcohol (stearyl alcohol) instead of fatty acid salt. The stearyl alcohol containing sample had peroxide decompose which induced bloating almost immediately. In contrast, the toothpaste composition of Table 1 was stable.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

What is claimed is:

1. A nonaqueous oral care composition comprising:
an orally acceptable vehicle;
a whitening agent; and
one or more gelling agents,
wherein the one or more gelling agents comprises a monovalent salt of a fatty acid, wherein the fatty acid is a C₁₂-C₃₂ fatty acid.

2. (canceled)

3. The oral care composition according claim 1, wherein the fatty acid is a saturated C₁₂-C₃₂ fatty acid.

4. The oral care composition according to claim 1, wherein the fatty acid is an unsaturated C₁₂-C₃₂ fatty acid.

5. The oral care composition according to claim 4, wherein the unsaturated C₁₂-C₃₂ fatty acid has an iodine value of less than 5 or less than 1.

6. (canceled)

7. The oral care composition according to claim 1, wherein the fatty acid is selected from lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, linoleic acid, arachidonic acid, palmitoleic acid, oleic acid, and a combination of two or more thereof.

8. (canceled)

9. (canceled)

10. The oral care composition according to claim 1, wherein the salt of a fatty acid is selected from sodium palmitate, sodium stearate, potassium palmitate, potassium stearate, and a combination of two or more thereof.

11. The oral care composition according to claim 1, wherein the one or more gelling agents are present in an amount of from about 1.7 weight % to about 10 weight %, based on the total weight of the oral care composition.

12. The oral care composition according to claim 1, wherein the orally acceptable vehicle is selected from water, glycerin, propylene glycol, polyethylene glycol, and combinations thereof, wherein the orally acceptable vehicle is present in an amount of from about 5 weight % to about 80 weight %, based on the total weight of the oral care composition.

13. The oral care composition according to claim 1, wherein the orally acceptable vehicle further comprises a co-polymer of ethylene oxide and propylene oxide having a molecular weight of greater than 5000 Da.

14. (canceled)

15. The oral care composition according to claim 13, wherein the co-polymer of ethylene oxide and propylene oxide is present in an amount from 5 wt. % to 20 wt. %, based on the total weight of the oral care composition.

16. (canceled)

17. The oral care composition according to claim 1, wherein the whitening agent comprises hydrogen peroxide, a source of hydrogen peroxide, or a combination thereof, wherein the whitening agent is present in an amount of from about 1 weight % to about 10 weight %, based on the total weight of the oral care composition.

18. (canceled)

19. The oral care composition according to claim 17, wherein the source of hydrogen peroxide comprises a cross-linked PVP hydrogen peroxide complex, a peroxide of alkali and alkaline earth metals, an organic peroxy compounds, a peroxy acid, a salt thereof, or a mixture thereof.

20-23. (canceled)

24. The oral care composition according to claim 1, wherein the oral care composition further comprises from about 0.01 weight % to about 5.0 weight % of a fluoride ion source selected from stannous fluoride, sodium fluoride, potassium fluoride, sodium monofluorophosphate, fluorosilicate salts, such as sodium fluorosilicate and ammonium fluorosilicate, amine fluoride, ammonium fluoride, and combinations thereof, based on the total weight of the oral care composition.

25-27. (canceled)

28. The oral care composition according to claim 1, wherein the oral care composition comprises fatty alcohols in an amount of less than 5.0 weight %, based on the total weight of the oral care composition.

29. The oral care composition according to claim 1, wherein the oral care composition comprises water in an amount of less than 5.0 weight %, based on the total weight of the oral care composition.

30. The oral care composition according to claim 1, wherein the oral care composition further comprises from about 10 weight % to about 20 weight %, based on a total weight of the oral care composition, of an abrasive selected from sodium metaphosphate, potassium metaphosphate, calcium pyrophosphate, magnesium orthophosphate, trimagnesium orthophosphate, tricalcium phosphate, dicalcium phosphate dihydrate, anhydrous dicalcium phosphate and the like, calcium carbonate, magnesium carbonate, hydrated alumina, silica, zirconium silicate, aluminum silicate including calcined aluminum silicate, polymethyl methacrylate, and combinations thereof.

31. (canceled)

32. The oral care composition according to claim 1, wherein the oral care composition further comprises from about 0.01% to about 1%, based on a total weight of the oral care composition, of an antioxidant selected from hydroxyanisole (BHA), butylated hydroxytoluene (BHT), vitamin A, carotenoids, vitamin E, flavonoids, polyphenols, ascorbic acid, herbal antioxidants, chlorophyll, melatonin, and combinations thereof.

33. (canceled)

34. (canceled)

35. The oral care composition according to claim 1, wherein the oral care composition further comprises one or more anionic surfactants selected from sodium lauryl benzene sulfonate, dodecyl sodium sulfoacetate, N-2-ethyl laurate potassium sulfoacetamide, sodium lauryl sarcosinate, sodium lauryl sulfate, and sodium ether lauryl sulfate, and combinations thereof.

36. (canceled)

37. The oral care composition according to claim 35, wherein the one or more anionic surfactants are present in an amount from about 0.03% to about 5% by weight, about 0.5% to about 3% by weight, about 1.0% to about 3% by weight, or about 2% to about 3% by weight, based on a total weight of the oral care composition.

38. A method for whitening teeth, comprising contacting a tooth surface of a subject in need thereof, with an oral care composition according to claim 1.
